(19) United States
(12) Patent Application Publication
(10) Pub. No.: US 2009/0042259 A1
(43) Pub. Date: Feb. 12, 2009

(54) Process for enzymatically converting a plant biomass

(75) Inventors: Bruce E. Dale, Mason, MI (US); Farzaneh Teymouri, Okemos, MI (US); Shishir Chundawat, East Lansing, MI (US); Venkatesh Balan, East Lansing, MI (US)

Correspondence Address: Ian C. McLeod;IAN C. McLEOD, P.C., 2190 Commons Parkway, Okemos, MI, 48864, US

(73) Assignee: Board of Trustees of Michigan State University, East Lansing, MI (US)

(21) Appl. No.: 12/221,972

(22) Filed: Aug. 8, 2008

Related U.S. Application Data
(60) Provisional application No. 60/964,102, filed on Aug. 9, 2007.

(51) Int. Cl. C12P 19/02

(52) U.S. Cl. 435/105

(57) ABSTRACT

The present invention describes a process for at least a 90% conversion of a plant biomass preferably by a reduction of the units of cellulase needed and by using a xylanase which acts synergistically with the cellulase to improve the yield of xylose and glucose as sugars. The process enables greater conversion of a lignocellulosic plant biomass to glucose and xylose for use as animal feeds and as fermentation as medium for producing ethanol.

### CROSS-REFERENCE TO RELATED APPLICATION(S)

This application claims benefit to U.S. Provisional Application Ser. No. 60/964,102, filed Aug. 9, 2007, which is incorporated herein by reference in its entirety.

### STATEMENT REGARDING FEDERALLY SPONSORED RESEARCH OR DEVELOPMENT

This work was supported by funds from the US Dept. of Energy under prime award #DE-FG36-04G014017 to Dartmouth College and from the State of Michigan ‘Research Excellence Fund’.

### BACKGROUND OF THE INVENTION

(1) Field of the Invention

The present invention relates to a process which uses both glucan (cellulose and stover) and cell wall degrading enzymes (eg. xylanase, xylosidase, pectinase) to degrade lignocellulosic material to produce glucose and xylose as sugars. The present invention particularly relates to a process for converting a lignocellulosic plant biomass comprising xylan and cellulose to xylose and glucose using a cellulase and a xylanase together. The yield of sugars comprising xylose and glucose is improved so that at least 90% by weight of these sugars in the plant biomass are recovered. The process is particularly effective when the plant biomass is pretreated with an AFEX process to expose the xylan and cellulose in the plant biomass. A preferred plant biomass is corn stover or the whole corn plant.

(2) Description of the Related Art

There is growing need to find replacements to petroleum, a depleting non-renewable resource, as the primary feedstock for the chemicals and fuels industry. Ethanol has many desirable features as a petroleum substitute and could help make a smoother transition from a petro-based to a bio-based chemical industry. Ethanol is produced in large quantities from natural resources like corn grain and sugarcane juice. However, there is need to find an inexpensive and widely available lignocellulosic source of biomass to avoid feedstock conflict with the prevalent food industry.

The increase in the price of petroleum feedstock has created opportunities for the development of combined biological and chemical processes that can provide liquid fuels and chemicals from alternate feedstock such as lignocellulosic plant biomass, particularly crop residues, forage crops and woody crops. Lignocellulosic materials are made up of three major organic fractions, cellulose, hemicellulose and lignin. Cellulose and hemicellulose together compose about 65-75% of overall biomass composition and can be converted to sugars.

There is approximately 252 million tonnes of corn stover available in the United States, of which, approximately 100 million tonnes is available for bioconversion; assuming 30% of the corn stover is left on the field for erosion control. (See e.g., Rooney T. 1998. Lignocellulosic Feedstock Resource Assessment. Report SR-580-24189. Golden, Colo.: National Renewable Energy Laboratory. p. 123.) Corn stover is the most abundant agricultural residue produced in the US every year, making it a prospective substrate for bioethanol production. Lignocellulosic compositional recalcitrance is one of the primary impediments in the successful implementation of a corn stover based biorefinery. Corn stover composition can vary with season, location, climatic conditions, phenotype etc., all of which play an important role in determining its cellulosic, hemicellulosic and lignin content. Recent economic analysis shows that a variation of 1% (of total dry matter) in the carbohydrate content of corn stover would change the minimum ethanol-selling price (MESP) by $0.018/gal, further highlighting the importance of substrate composition on the economics. (See e.g., Ruth M F, Thomas S R. May 4^{th} 2003; The Effect of Corn Stover Composition on Ethanol Process Economics; (Available online http://www.eere.energy.gov/biomass/pdfs/34040.pdf).) Determining the key compositional parameters of the plant cell wall that augment enzymatic hydrolysis would help in “designing” an ideal feedstock for the biorefinery. Re-engineering of plant cell wall composition could make the biomass more amenable to hydrolysis. (See e.g., U.S. Pat. No. 6,969,784 to Chiang et al.; U.S. Pat. Appln. 2006/0005270 to Dunn-Coleman et al.; and Harvey I M, McAllan A B, Theodorou M K, Beever D E. 1988. Phenolics in fibrous crop residues and plants and their effects on the digestion and utilisation of carbohydrates and proteins in ruminants. In Plant breeding and the nutritive value of crop residues. Proceedings of a workshop held at ILCA, Addis Ababa, Ethiopia, 7-10 Dec. 1987. ILCA, Addis Ababa.) However, there is still a lack of basic understanding on the role of corn stover composition on its pretreatment and enzymatic hydrolysis.

There are two main approaches to convert the complex plant cellulosic and hemicellulosic carbohydrates into simple sugars such as glucose and xylose: (1) acid hydrolysis; and (2) enzymatic hydrolysis. Acid hydrolysis is relatively inexpensive and simple but there are several major drawbacks associated with this process including: degradation of sugars due to the high temperature, formation of fermentation inhibitors, such as furfural, and expensive construction materials in equipment to handle the acids. The enzymatic hydrolysis of lignocellulosic plant biomass can be more attractive because of its specificity and absence of competitive degradation, which results in higher yields of sugars. However, enzyme costs can be prohibitively high and it is important to reduce the costs of enzyme production and/or to reduce the amount of enzyme used.

In the last three decades, a great deal of effort has been made to replace acid hydrolysis with enzymatic hydrolysis. In enzymatic hydrolysis, to achieve reasonable rate and yield, a biomass pretreatment step is necessary to render or disrupt the cellulosic structure of biomass and make it more accessible. Ammonia fiber explosion (AFEX) is an established pretreatment that makes the plant biomass more susceptible to enzymatic attack by increasing the digestibility of the plant biomass. (See e.g., U.S. Pat. No. 4,600,590 to Dale.) In the AFEX process, biomass is treated with ammonia under pressure and moderate temperatures for a few minutes followed by an explosive release of the pressure. The result is cellulose decrystallization, hemicellulose prehydrolysis, increased accessible surface area, and an alteration of lignin structure, which all together increase the digestibility of the plant biomass. Enzymatic (cellulase) hydrolysis of AFEX treated corn stover showed that under preferred AFEX treatment conditions, almost 95% conversion of cellulose to glucose was achievable; however, the conversion of hemicellulose (xylan) to xylose was only about 75%. The commercially available cellulase used contained only about 1% of its protein as xylanase activity.

Examples and descriptions related to AFEX process for treatment of plant material are described in the following patents or applications which are incorporated by reference herein in their entireties for all purposes: Provisional Application No. 60/936,509, filed Jun. 20, 2007; PCT Application Nos. PCT/U.S.07/10410, filed Apr. 30, 2007 and PCT/U.S.07/10415, filed Apr. 30, 2007; U.S. application Ser. No. 11/729,632, filed Mar. 29, 2007; U.S. Pat. Nos. 6,106,888 to Dale et al. and 6,176,176 to Dale et al.

The following references are related to pretreatment of plant biomass with ammonia to disrupt the cell biomass and are incorporated by reference herein in their entireties for all purposes: U.S. Pat. No. 4,600,590 to Dale; U.S. Pat. No. 4,644,060 to Chou; U.S. Pat. No. 5,037,663 to Dale; U.S. Pat. No. 5,171,592 to Holtzapple et al.; U.S. Pat. No. 5,865,898 to Holtzapple et al.; U.S. Pat. No. 5,939,544 to Karstens et al.; U.S. Pat. No. 5,473,061 to Bredereck et al.; U.S. Pat. No. 6,416,621 to Karstens; Felix, A., et al., Anim. Prod. 51, 47-61 (1990); and Waiss, A. C., Jr., et al., Journal of Animal Science, 35 No. 1, 109-112 (1972).

Many of the present-day biomass pretreatments (ethanol organosolv pretreatment, dilute acid pretreatment, ammonia recycle percolation) fractionate the various biomass components (lignin, hemicellulose & cellulose) into separate process streams. The removal of lignin and/or hemicellulose can substantially reduce the recalcitrance of biomass to enzymatic hydrolysis (See e.g., Wyman C E, Dale B E, Elander R T, Holtzapple M, Ladisch M R, Lee Y Y. 2005. Coordinated development of leading biomass pretreatment technologies. Bioresour Technol 96(18):1959-1966.) However, the pretreatment fractionation method is rather energy intensive and generates waste streams making it a significant bottleneck to an economical bioconversion process. AFEX pretreatment is an alkaline pretreatment process that effectuates a physico-chemical alteration in the lignocellulosic ultra and macro structure. Studies have shown that the AFEX pretreatment helps increase enzymatic digestibility several folds over the untreated Lignocellulosic. (See e.g., Teymouri F, Laureano-Perez L, Alizadeh H, Dale B E. 2005. Optimization of the ammonia fiber explosion (AFEX) treatment parameters for enzymatic hydrolysis of corn stover. Bioresour Technol 96(18):2014-2018.) AFEX pretreatment results in the decrystallization of cellulose (See e.g., Gollapalli L E, Dale B E, Rivers D M. 2002. Predicting digestibility of ammonia fiber explosion (AFEX) treated rice straw. Appl Biochem Biotech 98-100:23-35), partial depolymerization of hemicellulose, deacetylation of acetyl groups (See e.g., O'Connor J J. 1972. Tappi 55:353), cleavage of lignin carbohydrate complex (LCC) linkages, lignin C—O—C bond cleavage, increase in accessible surface area due to structural disruption (See e.g., Turner N D, McDonough C M, Byers F M, Holtzapple M T, Dale B E, Jun J H, Greene L W. 1990. Disruption of forage structure with an ammonia fiber explosion process. Proceedings, Western Section, American Society of Animal Sciences, Vol. 41) and increased wettability of the treated biomass (See e.g., Sulbaran de Ferrer B, Ferrer A, Byers F M, Dale B E, Aristiguieta M. 1997. Arch Latinoam Prod Anim 5 (Suppl. 1):112-114). The AFEX process demonstrates attractive economics compared to several leading pretreatment technologies based on a recent economic model for bioethanol from corn stover. (See e.g., Eggeman T, Elander R T. 2005. Process and economic analysis of pretreatment technologies. Bioresour Technol 96(18):2019-2025.)

The surface composition of AFEX treated lignocellulosic could be an important factor that affects the rate of enzymatic hydrolysis and microbial fermentation. Electron Spectroscopy for Chemical Analysis (ESCA) is an excellent surface chemical analysis technique that has been used in the past to identify the chemical composition of biomass surfaces. (See e.g., Dorris G M, Gray D G. 1978. The surface analysis of paper and wood fibers by ESCA, II. Surface composition of mechanical pulps. Cellul Chem Technol 12:721-734.) Most of the work done in ESCA studies has tended to focus on the analysis of wood pulp and fiber surfaces for the paper industry, with little focus on agricultural residues like corn stover. (See e.g., Laine J, Stenius P, Carlsson G, Strom G. 1994. Surface characterization of unbleached kraft pulps by means of ESCA. Cellulose 1:145-160.)

Fractionating lignocellulosic material based on its compositional and anatomical differences might be useful in separating the more recalcitrant fractions (i.e. cobs, stalk) from lesser recalcitrant portions (i.e. leaves, husk). Some work in the past has elucidated the influence of biomass fraction composition (See e.g., Alvo P, Belkacemi K. 1997. Enzymatic saccharification of milled timothy (Phleum pretense L.) and Alfalfa (Medicago sativa L.). Bioresour Technol 61:185-198.; and Hoskinson R L, Hess J R, Foust T D, McKean W T, Lewis M S. 2001. Fractionation of higher value crop residue components for conversion into bioenergy and industrial products. ASAE Paper 01-6077. Annual meeting of the American Society of Agricultural Engineers at Sacramento, Calif. 30 July-1 August, 2001, p. 9) and particle size (See e.g., Elshafei A M, Vega J L, Klasson K T, Clausen E C, Gaddy J L. 1991. The saccharification of corn stover by cellulase from Penicillium-funiculosum. Bioresour Technol 35(1):73-80) on pretreatment and enzymatic hydrolysis. It was shown that fractionating corn stover into cobs, husk-leaves and stalk improved the overall hydrolysis yields. (See e.g., Montross M D, Crofcheck C L. 2004. Effect of stover fraction and storage method on glucose production during enzymatic hydrolysis. Bioresour Technol 92:269-274.) It was found that the untreated leaf fraction gave the highest glucan conversion of 91%, whereas the untreated cobs and stalks had efficiencies of 63% and 33% respectively. The trend is rather intuitive since the stalk is composed largely of internodes rich in recalcitrant lignified xylem vessels. A pre-collection system at the farm/biorefinery to separate out the stover fractions, either pneumatically and/or by sieving, could be a possible alternative to reduce pretreatment and hydrolysis costs. The fractions could be pretreated separately to get optimum conversions as compared to a combined pretreatment/hydrolysis for the whole plant. The cob fraction could also be used to obtain other value added products, such as xylooligosaccharides. (See e.g., Garrote G, Dominguez H, Parajo J C. 2002. Autohydrolysis of corncob: study of non-isothermal operation for xylooligosaccharide production. J Food Eng 52(3): 211-218; and Yang R, Xu S, Wang Z, Yang W. 2005. Aqueous extraction of corncob xylan and production of xylooligosaccharides. LWT-Food Sci Technol 38(6):677-682.) However, there is lack of data on AFEX pretreatment and enzymatic hydrolysis to evaluate the technical and economic feasibility of fractionating corn stover into various fractions (i.e. cobs, leaves, husk and stalk) for ethanol production.

Diffuse reflectance infra-red Fourier transform (DRIFT) spectroscopy has been used to quantify chemical changes that take place in lignocellulosics after pretreatment, including AFEX. (Laureano-Perez L. 2005. Spectroscopic and chemical characterization of biomass. Doctoral Dissertation, Michigan State University.) Most of the conventional IR spectroscopy techniques are time consuming, cumbersome and suffer from reproducibility problems given the complexity of sample preparation. With the development of the Attenuated Total Reflectance (ATR) technique most of these issues can be alleviated. FTIR-ATR measures the absorption of the evanescent wave that penetrates the sample (0.5-5 micron penetration) when the incident light is reflected at the crystal/solid interface.

### OBJECTS

It is therefore an object of the present invention to provide a process which enables a higher yield of xylose along with glucose from a plant biomass. It is also an object of the present invention to reduce the amount of cellulase required for a hydrolysis process. It is further an object of the present invention to provide an improved treated plant biomass as an animal feed.

These and other objects will become increasingly apparent to those skilled in the art by reference to the following description and drawings.

### SUMMARY OF THE INVENTION

The present invention provides a process for converting disrupted lignocellulosic plant biomass to sugars comprising xylose and glucose, the process comprising: (a) providing a plant biomass comprising: (i) cell walls comprising xylans, glucans and pectins; and (ii) glucans inside the cell walls; and (b) adjusting a ratio of enzymatic activity of a glucanase to a hemicellulase so that at least 90% by weight of available cellulose and the xylans in the cell wall of the plant biomass are converted to the sugars. In further embodiments, the plant biomass material is corn stover. In still further embodiments, the ratio of filter paper cellulase units (FPU) as the glucanase to units of hemicellulase is between about 10 to 1 and 2 to 1. Still further, the plant biomass is treated with an AFEX process step to disrupt the plant biomass. Further still, a reduced amount of the glucanase and the hemicellulase over an amount of each enzyme alone needed to achieve the 90% by weight conversion is used for the converting. Further, the biomass is washed in a liquid after an AFEX process step to remove phenolics in the liquid. Further still, the preferred process is with a loading of 7.5 FPU of cellulase as the glucanase and xylanase as the hemicellulase per gram of available glucans and xylans. Further, preferably 5 to 15 International units of xylanase as a hemicellulase and 5 to 15 filter paper units of cellulase as the glucanase are used to achieve the 90% by weight conversion. Further still, a weight of units of the cellulase as the glucanase with xylanase as the hemicellulase per gram of cellulose and xylan in the plant biomass is less than a weight of units of cellulase alone to achieve the 90% by weight conversion.

In an exemplary embodiment, the corn stover is pre-milled to reduce particle size of the biomass to be between 0.05 mm to 0.85 mm. In a further embodiment, step (b) further comprises adjusting a ratio of enzymatic activity by providing a pectinase in addition to the glucanase and the hemicellulase. Still further, the hemicellulase comprises at least one of xyloglucanase, β-xylosidase, endoxylanase, α-l-arabinofuranosidase, α-glucuronidase, and acetyl xylan esterase. In a further embodiment, the plant biomass is treated with an AFEX process step to expose the xylans and cellulose and then milled to a reduced particle size. In a further exemplary embodiment, the AFEX treatment comprises the steps of: (a) providing the biomass with 60% moisture to a high-pressure reactor with liquid ammonia to a vessel; (b) raising and maintaining the temperature of the vessel to 90° C. for up to five minutes; and (c) explosively relieving the pressure to cause a pressure drop such that the ammonia vaporizes causing explosive decompression of the biomass and fiber disruption.

The present invention relates to an improvement in a process for converting a disrupted lignocellulosic plant biomass comprising xylan and cellulose to xylose and glucose, which comprises: adjusting a ratio of enzymatic activity of cellulase to hemicellulase so that at least 90% by weight of available cellulose and xylan in the plant biomass is converted to glucose and xylose. In further embodiments, the plant biomass material is corn stover. In still further embodiments, ratio of filter paper cellulase units (FPU) for cellulase to units of hemicellulase is between about 10 to 1 and 2 to 1. Still further, the plant biomass is treated with an AFEX process step to expose the xylan and cellulose. In still further embodiments, a reduced amount of cellulase with hemicellulase and an amount of cellulase alone needed to achieve the 90% by weight conversion is used for the converting. Still further, the biomass is washed after an AFEX process step to remove a liquid comprising phenolics. Further still, the preferred process is with a loading of 7.5 FPU of cellulase and hemicellulase per gram of available glucan and xylan. Further, preferably 5 to 15 International units of hemicellulase to xylan loadings and 5 to 15 filter paper units of cellulase are used to achieve the 90% by weight conversion. Finally, a weight of units of the cellulase with hemicellulase per gram of glucan and xylan in the plant biomass is less than a weight of units of cellulase alone to achieve the 90% by weight conversion.

In an exemplary embodiment, the corn stover is pre-milled to reduce particle size of the biomass. In a further embodiment, the particle size is reduced to be between 0.05 mm to 0.85 mm. Still further, the particle size is reduced to be between 0.15 mm to 0.5 mm. In a further embodiment, the plant biomass is treated with an AFEX process step to expose the xylan and cellulose and then milled to a reduced particle size. In a further exemplary embodiment, the AFEX treatment is a pretreatment to the biomass prior to enzymatic hydrolysis comprising the steps of: (a) providing the biomass with 60% moisture to a high-pressure reactor with liquid ammonia to a vessel; (b) raising and maintaining the temperature of the vessel to 90° C. for up to five minutes; and (c) explosively relieving the pressure to cause a pressure drop such that the ammonia vaporizes causing explosive decompression of the biomass and fiber disruption.

The present invention also provides a process for converting disrupted lignocellulosic plant biomass to sugars comprising xylose and glucose, the process comprising: (a) providing a plant biomass comprising: (i) cell walls comprising xylans, glucans and pectins; and (ii) glucans inside the cell walls; and (b) adjusting a ratio of enzymatic activity of a glucanase to a cell-wall degrading lyase enzyme so that at least 90% by weight of available cellulose and the xylans in the cell wall of the plant biomass are converted to the sugars. In a further embodiment, the cell-wall degrading lyase enzyme comprises at least one of a hemicellulase and a pectinase. In a still further embodiment, the hemicellulase comprises at least one of xyloglucanase, β-xylosidase, endoxylanase, α-l-arabinofuranosidase, α-glucuronidase, and acetyl xylan esterase. Further, the pectinase comprises at least one of pectate lyase, polygalacturonase, and pectin esterase.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic flow chart showing water washing and soluble recovery procedures. UCS, untreated corn stover; ACS, AFEX-treated corn stover, BM, biomass.

FIG. 2A illustrates various parts of corn plants and a pie chart representing mass distributions of an exemplary corn plant.

FIG. 2B shows various fractions for ground sample. Percent mass distributions of various particle size fractions for premilled (PM) and ground corn stover are shown as a pie chart.

FIG. 3A is a graph showing exemplary trends for chemical changes in biomass after AFEX using FTIR-ATR (i) PM AFEX-treated corn stover, and (ii) PM Untreated stover.

FIG. 3B is a graph showing FTIR data for stover fractions (a) #35, (b) #40, (c) #70, (d) #80, (e) #100, and (f) #120. Unprimed and primed symbols represent AFEX-treated and untreated samples, respectively.

FIG. 4A is a graph showing a phenolic content of AFEX-treated and untreated corn stover wash streams: (□) Untreated stover % gallic acid; ( [symbol] ) Untreated stover % ferulic acid; (□) AFEX-treated stover % gallic acid; and ( [symbol] ) AFEX-treated stover % ferulic acid. Calibration curfes for ferulic acid (▪) and gallic acid (▴) absorbance are shown on the top right corner as an insert.

FIG. 4B is a graph showing ferulic acid content of wash stream after single wash (UU-1W: Uncut corn stover→Untreated→Single Wash and UAG-1W: Uncut PM corn stover→AFEX→Ground to 0.08 mm→Single wash), multiple wash (UAG-3W: Uncut corn stover→AFEX→Ground to 0.08 mm→Washed three times), and after size reduction (GA-1W: ground to 0.08 mm→AFEX→Single wash).

FIG. 4C shows wash stream recovered for AFEX-treated (left) and untreated corn stover (right).

FIGS. 5A and 5B are graphs showing the effect of size reduction and water washing of (A) untreated corn stover and (B) AFEX-treated corn stover on 72-hour enzymatic hydrolysis yields; with and without 10% xylanase supplementation on glucan (G) and xylan (X) conversions. (Abbreviations: CS, corn stover; U-GBA, unwashed ground before AFEX; W-GBA, washed ground before AFEX; U-GM, unwashed ground after AFEX; W-GAA, washed ground after AFEX. The standard error bars are based on duplicate experiments.)

FIGS. 6A and 6B show 72 (□▪) and 168 ( [symbol] )-hour glucan conversion for unwashed (Ia) and washed (IIa) untreated/AFEX-treated stover fractions; with (+) and without (−) supplemental xylanase loading. The standard error bars are based on duplicate experiments.

FIGS. 7A and 7B show 72 (□▪) and 168 ( [symbol] )-hour xylan conversions for unwashed (Ib) and washed (IIb) untreated/AFEX-treated stover fractions; with (+) and without (−) supplemental xylanase loading. The standard error bars are based on duplicate experiments.

FIG. 8A shows ESCA spectra lying within binding energy range (0-1,000 eV) for untreated corn stover.

FIG. 8B shows deconvoluted peaks of C1s spectra for untreated corn stover.

FIG. 9 graphically shows the effect of varying cellulase, xylanase and pectinase loading for AFEX treated corn stover (90° C., 1:1 ammonia:biomass loading, 60% moisture (dwb)). Experiment was done using ground biomass in a micro plate with 1% glucan loading, 750 μl reaction volume, 375 RPM. The reaction was done at 50° C. for a period of 6 hrs. From the figure it is clear that both pectinase and xylanase activities along with cellulase improves glucan conversion.

FIGS. 10A, 10B, 10C, and 10D graphically shows synergistic hydrolysis of untreated and AFEX treated poplar for 3 and 24 hours. The experiments were done in a micro plate with 1% glucan loading. The enzymatic hydrolysis done at 50° C. for a period of 3 and 24 hrs respectively. Here we could see that for a lower cellulase loading, both xylanase or pectinase xypplimentation improves glucan conversion.

### DESCRIPTION OF THE PREFERRED EMBODIMENTS

The term “cellulase” is synonymous with plant cellulose polysaccharide degrading enzymes. A partial list of such enzymes is set forth in U.S. Pat. No. 6,818,803 B1 which is incorporated herein by reference in its entirety. The enzymes are in Engine Classification EC 3.2.1x.

The term “plant cell wall degrading enzyme” includes a “lyase” and refers to enzymes which degrade lignins, xylans, glucans, pectins and other components of the cell wall. The term “lyase” means an enzyme which degrades a polysaccharide at carbon oxygen bonds, one such enzyme is pectin lyase for instance (E 4.2.10).

The term “hemicellulase” comprises at least one of xyloglucanase, β-xylosidase, endoxylanase, α-l-arabinofuranosidase, α-glucuronidase, and acetyl xylan esterase. This is a cell wall component.

The term “disrupted” means to break apart the cell walls of the plant material to enable the release of structural carbohydrates and other cell wall polymers.

The term “plant” means the biomass of a monocot and dicot leafy plants in whole or in part.

The term “glucanase” refers to an enzyme which breaks down a glucan in the cell wall or in the cell.

The hydrolysis of glucan and xylan can be characterized and closely linked to whatever enhances glucan conversion also tends to increase xylan conversion (and vice versa). It is desirable to improve the balance between cellulase and xylanase activities in the hydrolysis mixture. Given the number and variety of different chemical linkages associated with hemicellulose, the close chemical and physical interactions of all three major plant biomass components and the modifications of these chemical and physical interactions caused by an AFEX treatment, it is important to determine not only adequate total hemicellulase activity but also the correct distribution of enzyme activities within the hemicellulase complex. The present disclosure provides for an exemplary combination of cellulase and xylanase for hydrolysis of AFEX treated biomass. In an exemplary embodiment, the present disclosure is illustrated by developing and optimizing this mixture of enzyme activities for AFEX treated corn stover. The optimization results in at least 90% conversion in both cellulose and hemicellulose preferably using an overall loading of 7.5 Filter Paper Units (FPU) of (cellulase+xylanase) per gram of available (glucan+xylan). This composition allows a trade off of total cellulase and xylanase activities, which in turn, reduces the total enzyme loadings in enzymatic hydrolysis.

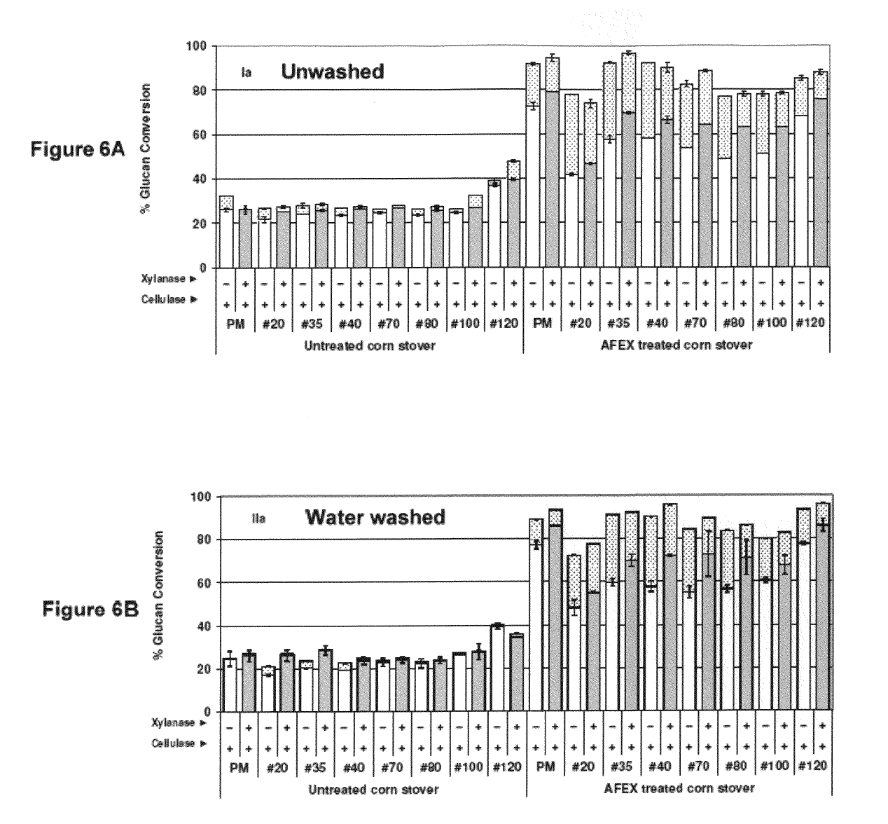

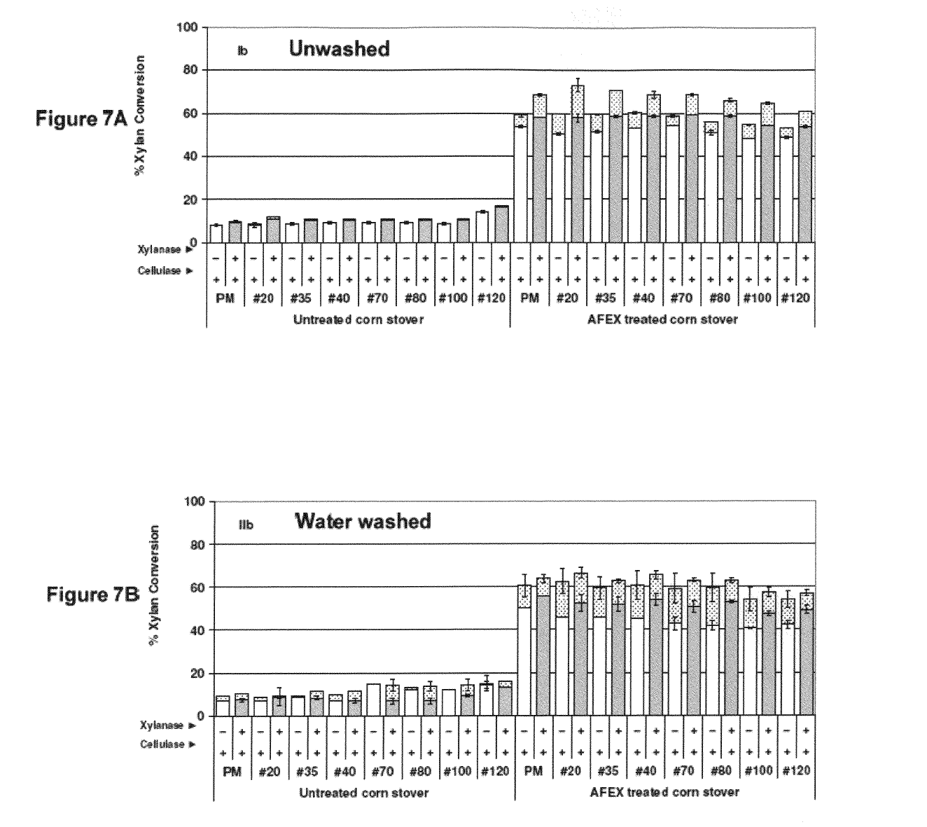

Commercially available xylanase was used in combination with the cellulase mixture and the resulting effects on both glucose and xylose yields in enzymatic hydrolysis of AFEX treated corn stover was determined. The treatment started with similar xylanase to xylan loadings (5-15 International Units per gram of xylan) as used in glucan hydrolysis (5-15 FPU per gram of glucan) with reference to National Renewable Energy Laboratory (NREL, Golden, Colo., LAP-006). When xylose yields exceeded 90% of theoretical, the cellulase loading was dropped by half because it was found that the added xylanase increased the potency of available cellulase. These results were evaluated to minimize total mass of enzyme added to produce the maximum amount of sugars generated. The results are shown in FIGS. 6A and 6B for glucan conversion and FIGS. 7A and 7B for xylan conversion. The treatments preferably included a pre-soak with water to enhance the composition of xylose and glucose production by removing inhibitors such as phenolics from the treated plant biomass. Cellulase activities in combination with pretreatment conditions were examined until the lowest possible enzyme dosage giving the maximum grams of sugar is produced. Better results were achieved in terms of total mass of sugars produced at the same total amount of enzyme (in total mass of xylanase and cellulase added) by optimizing cellulase and xylanase levels in comparison with enzymatic (cellulase only with minimal xylanase) hydrolysis of AFEX treated corn stover.

FIGS. 6A and 6B show percent conversion of glucan as a result of treatment of unwashed corn stover with and without an AFEX treatment and with and without water washing after the AFEX treatment. Both xylanase and cellulase were used in some of the experiments. It is clear that the AFEX process with water washing produces much higher yields of glucose and xylose particularly with xylanase and cellulase. This was also true for xylan conversion to xylose in FIGS. 7A and 7B. FIGS. 7A and 7B show a significant improvement in the xylose and glucose production from cellulose and xylan in the presence of both cellulase and xylanase together.

Reference is now made to the following examples to further illustrate particular aspects of the present disclosure. In now way are the following exemplary embodiments intended to limit the scope of the present disclosure thereto. Thus it should be apparent to one having skill in the technical art that variations and modifications to the following disclosure are within the scope of the disclosure.

### EXAMPLE

Particle size and compositional variance are found to have a substantial influence on ammonia fiber explosion (AFEX) pretreatment and enzymatic hydrolysis of lignocellulosic biomass. Corn stover was milled and fractionated into particle sizes of varying composition. The larger particle size fractions (rich in corn cob and stalk portions) were found to be more recalcitrant to hydrolysis compared to the smaller size fractions (rich in leaves and husk portion). Electron spectroscopy for chemical analysis (ESCA) and Fourier transform infrared spectroscopy (FTIR) were used for biomass surface and bulk compositional analysis respectively. The ESCA results showed a 15-30% decrease in the O/C (oxygen to carbon) ratio after the pretreatment indicating an increase in the hydrophobic nature of biomass surface. FTIR results confirmed cleavage of the lignin-carbohydrate complex for the AFEX treated fractions. The spectroscopic results indicate the extraction of cleaved lignin phenolic fragments and other cell wall extractives to the biomass surface upon AFEX. Water washing of AFEX treated fractions removed some of the hydrophobic extractives resulting in a 13% weight loss (dry weight basis). Phenolic content of wash stream was evaluated by the modified Prussian blue method. Removal of ligno-phenolic extractives from the AFEX treated biomass by water washing vastly improved the glucan conversion as compared to the unwashed samples. Reduction in substrate particle size was found to affect the AFEX process and rate of hydrolysis as well. Implications of the stover particle size, composition and inhibitory role of the phenolic fragments on an integrated biorefinery are described in greater detail below.

### INTRODUCTION

The effect of grinding corn stover and fractionating it into various particle size ranges (untreated/AFEX treated) on its composition and rate of enzymatic hydrolysis was examined. The effect of water washing the AFEX treated fractions on enzymatic hydrolysis was further explored. The role of AFEX pretreatment on the ultra structural level is better understood using novel analytical techniques, ESCA (surface analytical technique) and modified Prussian blue method. FTIR-ATR spectroscopy is used to qualitatively determine the chemical changes in the lignocellulosic biomass upon AFEX pretreatment.

### MATERIALS AND METHODS

### Biomass Milling and Sieving

Pre-milled (PM) corn stover, passed through a 10 mm screen, was generously provided by the National Renewable Energy Laboratory (NREL), Golden (Colo.). The corn stover was milled in a conventional laboratory blender for a period of ten minutes, followed by sieving for twenty minutes in a Ro-Tap testing sieve shaker (Model B, Tyler) to obtain various corn stover fractions. The PM corn stover was also milled down to a desired particle size range using a centrifugal mill (Model ZM 200, Retsch) fitted with various ring sieve attachments.

### Near Infra-Red (NIR) Compositional Analysis

The compositional analysis of the corn stover fractions was performed by the Near-IR spectroscopic analysis (courtesy Dr. Bonnie Hames) at the National Renewable Energy Laboratory (NREL), Golden (Colo.). The Near-IR spectroscopy method is a rapid and inexpensive method to determine the composition of biomass as compared to the standard wet chemical methods. (See e.g., Hames B R, Thomas S R, Sluiter A D, Roth C J, Templeton D W. 2003. Rapid biomass analysis—New tools for compositional analysis of corn stover feedstocks and process intermediates from ethanol production. Appl Biochem Biotech 105:5-16.)

### AFEX Pretreatment

Corn stover was pretreated by the AFEX pretreatment process. The biomass with 60% moisture (kg water/kg dry biomass) was transferred to a high-pressure Parr reactor and liquid ammonia (1 kg of ammonia/kg of dry biomass) was slowly charged to the vessel. The temperature was raised and maintained at 90° C. for five minutes residence time at that temperature before explosively relieving the pressure. The instantaneous drop of pressure in the vessel caused the ammonia to vaporize, causing an explosive decompression of the biomass and considerable fiber disruption. The pretreated material was allowed to stand under a hood overnight to remove the residual ammonia and stored in a freezer until further use.

Water Washing of Untreated/AFEX treated Biomass

Untreated and AFEX treated biomass were presoaked and washed in distilled (de-ionized) water with a substrate to water loading of 1:10 (w/w). The slurry was mixed for 15 minutes. The wash liquid was removed from the substrate by squeezing the slurry through a filtration cloth and stored in the refrigerator for further analysis. The washed substrates were hydrolyzed immediately to prevent microbial growth. The moisture content of the washed substrate was determined using a moisture analyzer (Model MF-50, A&D).

### Mass Balance for Water Washing

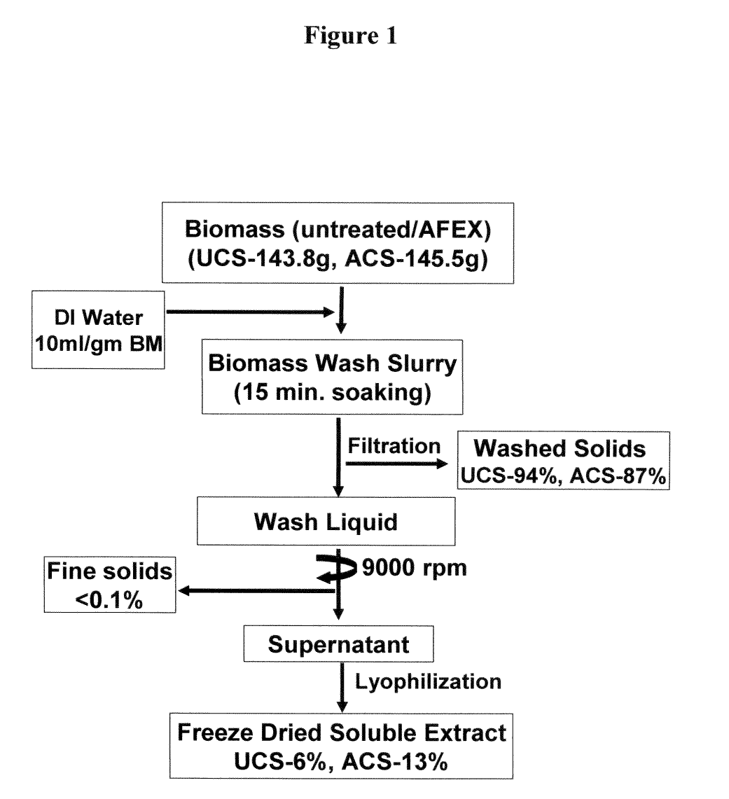

A mass balance for determining the solids and soluble content of the wash liquid stream was carried out according to the procedure (i.e., schematic flow chart) outlined in FIG. 1. Untreated (143.8 grams dry BM) and AFEX (145.5 grams dry BM) treated biomass were soaked and stirred in distilled (de-ionized) water with a substrate to water loading of 1:10 for 15 minutes. The wash liquid was removed from the substrate by filtering the slurry through a filtration cloth and centrifuged at 9000 rpm to remove fine solid particles from the wash stream. The supernatant was lyophilized to recover the washed soluble from the biomass for further characterization. The moisture content of the sample was determined using a lab oven at 105° C.

### Enzymatic Hydrolysis

The NREL standard protocol (LAP-009) was followed for enzymatic hydrolysis of the biomass. Cellulase (Spezyme CP) and xylanase (Multifect™) enzymes were a generous gift from Genencor International (Rochester, N.Y.). The substrate was hydrolyzed at a glucan loading of 1% (w:v) in a 0.05 molar citrate buffer solution (pH 4.8) at the desired cellulase enzyme loading (protein concentration 123 mg/ml) of 15 FPU/gm glucan and β-glucosidase loading of 64 pNPGU/gm glucan. Xylanase (protein concentration 42 mg/ml) supplementation was carried out at 10% of the total milligrams of cellulase protein loaded. The protein concentration of the enzymes was determined by the BCA protein assay. Samples were hydrolyzed at 50° C. with gentle agitation (90 rpm) for a period of 168 hours. The hydrolyzed samples were boiled to denature the enzymes and filtered through a 0.2 micron nylon membrane filter at predetermined time periods (72 and 168 hours). The samples were frozen for subsequent HPLC sugar analysis.

### HPLC Sugar Analysis

A high performance liquid chromatography (HPLC) system was used for sugar analysis. The HPLC system consisted of Waters Pump and Waters 410 refractive index detector, an Aminex HPX-87P carbohydrate analysis column equipped with a deashing guard cartridge. Degassed HPLC grade water was used as the mobile phase at 0.6 ml/min at a column temperature of 85° C. The injection volume was 20 μl with a run time of 20 min. Mixed sugar standards were used for quantification of cellobiose and other monosaccharides (glucose, xylose, galactose, arabinose and mannose) in the samples.

### FTIR-ATR Spectroscopic Analysis

Spectrum One FTIR system (Perkin Elmer) with a universal ATR (Attenuated Total Reflection) accessory was used to qualitatively monitor chemical changes in the AFEX pretreated biomass. The sample was pressed uniformly and tightly against the diamond surface using a spring-loaded anvil. Mid-IR spectra were obtained by averaging 16 scans from 4000 to 400 cm⁻¹ at 2 cm⁻¹ resolution. The region between 1900 and 2200 cm⁻¹ was avoided because of the strong diamond IR absorption. Baseline and ATR corrections for penetration depth and frequency variations were carried out using the Spectrum One software supplied with the equipment.

### ESCA Analysis

The ESCA surface chemical characterization was carried out using a Physical Electronics PH15400 ESCA electron spectrometer equipped with a non-monochromatic Mg Kα (15 V, 300W) X-ray source. An area of 250×250 square microns with a take off angle of 45 degrees was analyzed. Peak intensities were determined by peak area integration. Curve fitting to the C1s and O1s peaks was carried out with a Lorentzian-Gaussian curve-fitting program. Calcium and nitrogen peaks were deconvoluted and fitted by the software.

### Modified Prussian Blue (MPB) Method

Phenolic compound content of the wash stream was quantified by the MPB method (See e.g., Graham H D. 1992. Stabilization of the Prussian blue color in the determination of polyphenols. J Agric Food Chem 40:801-805.) The wash stream was centrifuged to remove suspended solids and filtered through a 0.2-micron nylon membrane filter before carrying out the MPB analysis. Phosphoric acid (85%), Gum Arabic, Gallic acid, Ferulic acid, K₃Fe(CN)₆ and FeCl₃, were purchased from the Sigma Chemical Co. (St. Louis, Mo.). MPB stabilizer reagent was prepared by mixing 6 parts of distilled water with 2 parts of 85% phosphoric acid and 2 parts of 1% gum arabic solution. One hundred micro liters of the undiluted wash extract was placed in duplicate sets of large (150×25 mm) falcon tubes. Three milliliters of distilled (deionized) water was added to the tubes and mixed well with the wash extract. One (1) ml of 0.016 M K₃Fe(CN)₆ was added followed immediately by 1 ml of 0.02 M FeCl₃ in 0.1 N HCl. The mixture was vortex mixed and allowed to stand at room temperature. After 15 min, 5 ml of the MPB stabilizer reagent was added to the contents of the tube and vortexed again. The intensity of the Prussian blue color developed was measured at 700 nm against a reagent blank using a digital spectrophotometer (Barnstead/Thermolyne Series SP-800). Gallic and ferulic acids were used as standards to determine equivalent phenolic concentration.

### RESULTS AND DISCUSSIONS

### Milling and Sieving Data

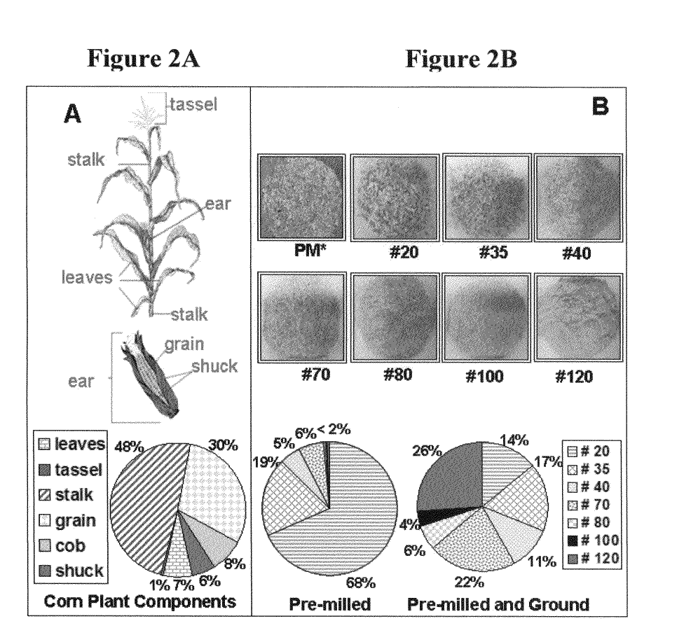

FIG. 2A illustrates photographically various parts of a corn plant and exemplary mass distributions by way of the pie chart. FIG. 2B shows corn stover fractions obtained upon laboratory blender milling resulted in the following particle size ranges: a) Fraction 20-Mesh (>850 microns), b) Fraction 35-Mesh (850-500 microns), c) Fraction 40-Mesh (500-425 microns), d) Fraction 70-Mesh (425-212 microns), e) Fraction 80-Mesh (212-180 microns), f) Fraction 100-Mesh (180-150 microns), and g) Fraction 120-Mesh (<150 microns). FIG. 2B also illustrates particle size percentages with reference to the pie charts for both pre-milled and pre-milled and ground.

The average particle size (5 replicates with variation under 1-2% from average) distribution of the milled material as compared to pre-milled (PM) corn stover is shown in FIG. 2B. The PM stover has a more skewed particle size distribution tending towards the larger sizes. However, the particle size distribution for the milled stover fractions is approximately normally distributed centered about mid-range mesh cut (425-212 microns).

Corn stover produces around 25-30% of leafy fraction (leaf-husk-sheaths) and 70-75% of fibrous and hard material (stalk and cobs), as shown in the pie chart of FIG. 2A. The more recalcitrant stalk could be preferentially screened from the leaf and cob fraction during the harvest itself and be left behind on the fields to control erosion and increase soil organic matter (See e.g., Montross M D, Crofcheck C L. 2004. Effect of stover fraction and storage method on glucose production during enzymatic hydrolysis. Bioresour Technol 92:269-274). The leaf and cob fraction could be hydrolyzed using less severe AFEX pretreatment conditions as compared to the stalk portion, reducing pretreatment costs.

### Bulk Component Analysis of Corn Stover Fractions

Compositional analysis of corn stover fractions (Table 1) provides for a good indication of the possible origin of each fraction. The larger particle size fractions have a higher hemicellulosic content and lower water soluble content compared to PM corn stover. The finer fractions are richer in water-soluble components (i.e. proteins, soluble sugars) and have a lower hemicellulosic content. The larger fractions are more likely to be derived from the stem portion as compared to the finer fractions that are likely from the leaf and broken midrib and veins of the stover (Alvo et al., 1997).

TABLE 1
Composition of various particle size fractions of pre-milled (PM) and ground corn stover

| | | # 20 | # 35 | # 40 | # 70 | # 80 | # 100 | # 120 |
|---|---|---|---|---|---|---|---|---|
| Components | Pre-milled | (>850 μm) | (850-500 μm) | (500-425 μm) | (425-212 μm) | (212-180 μm) | (180-150 μm) | (<150 μm) |
| Glucan | 32.6 | 33.5 | 34.4 | 34.4 | 34.0 | 34.0 | 33.3 | 32.3 |
| Xylan | 23.4 | 27.5 | 24.7 | 23.5 | 22.6 | 21.9 | 21.9 | 20.1 |
| Galactan | 1.4 | 1.0 | 1.3 | 1.3 | 1.4 | 1.5 | 1.7 | 1.8 |
| Arabinan | 2.7 | 2.4 | 2.7 | 2.5 | 2.6 | 2.7 | 2.9 | 2.9 |
| Mannan | 0.3 | 0.7 | 0.4 | 0.3 | 0.2 | 0.1 | 0.1 | 0.0 |
| Lignin | 12.3 | 12.5 | 12.4 | 12.6 | 12.6 | 12.9 | 12.8 | 12.0 |
| Protein | 3.1 | 1.8 | 2.0 | 2.3 | 2.7 | 2.9 | 3.3 | 4.3 |
| Water Solubles | 3.9 | 0.1 | 2.7 | 4.3 | 6.0 | 7.9 | 8.5 | 11.3 |
| Alcohol Solubles | 3.5 | 2.3 | 3.2 | 3.2 | 3.6 | 3.9 | 3.9 | 4.4 |

### FTIR-ATR Analysis

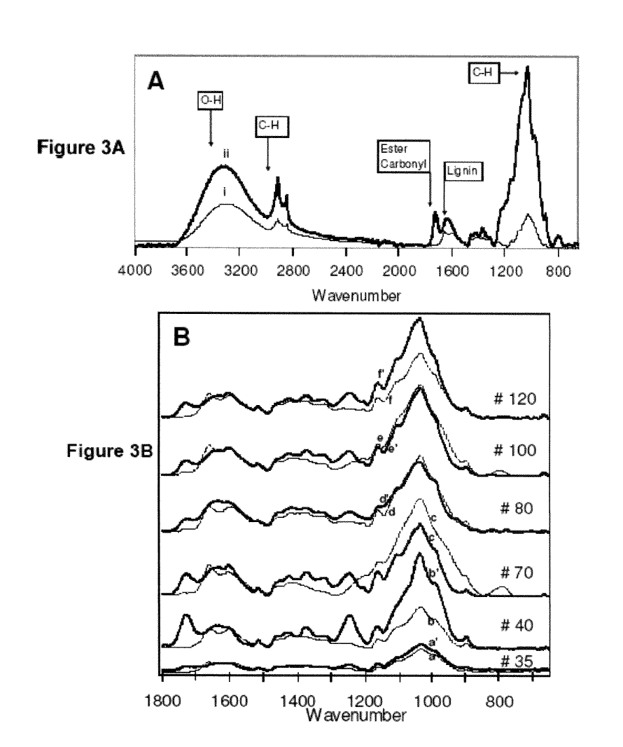

The FTIR spectra for AFEX pretreated stover fractions are shown in FIG. 3. The peaks around 3400 cm⁻¹ are attributed to the O—H stretch, while those at 900 cm⁻¹ are attributed to the antisymmetric, out of phase ring stretch of amorphous cellulose. (See e.g., Michell A J. 1988. Usefulness of Fourier-transform infrared difference spectroscopy for studying the reactions of wood during pulping. Cellul Chem Technol. 22:105-113.) The peaks at 1599, 1508, and 1451 cm⁻¹ are from aromatic skeletal vibrations in the lignin; the 1112 and 1026 cm⁻¹ peaks are from aromatic C—H in-plane deformation; and the peak at 828 cm⁻¹ is from aromatic C—H out-of-plane bending. (See e.g., Stewart D, Wilson H M, Hendra P J, Morrison I M. 1995. Fourier-Transform Infrared and Raman Spectroscopic Study of Biochemical and Chemical Treatments of Oak Wood (Quercus rubru) and Barley (Hordeum vulgare) Straw. J Agric Food Chem 43:2219-2225.) The peak at 1510 cm⁻¹ is due to the aromatic ring stretch vibration in lignin and was used as the internal reference band to normalize the spectra for comparison. (See e.g., Gollapalli L E, Dale B E, Rivers D M. 2002. Predicting digestibility of ammonia fiber explosion (AFEX) treated rice straw. Appl Biochem Biotech 98-100:23-35.) Some of the important peaks identified from literature that can serve to illustrate the effect of the AFEX on the biomass compositions are ester carbonyl peak at 1720 cm⁻¹ and the aldehyde peak at 1640 cm⁻¹. These types of bonds are present in the hemicellulose and hemicellulose-lignin complexes. A decrease in these peaks is directly related to delignification and hydrolysis of hemicellulose respectively. There is also a change in the relative intensity of the peaks at 1670 and 1610 cm⁻¹, which are characteristic of amide linkages (See e.g., Silverstein R M, Bassler G C, Morrill T C. 1981. Spectrophotometric identification of organic compounds, 4^{th} Edn. Newyork: John Wiley and Sons. p 124), possibly due to the ammonolysis of the acetyl groups in hemicellulose (See e.g., O'Connor J J. 1972. Tappi 55:353).

FTIR-ATR spectroscopy had been a severely limited technique in the past due to the unavailability of hard crystals which were chemically inert and optically transparent in the visible and most of the mid-IR region. However, with the introduction of ATR cells utilizing diamond and silicon based crystals, this technique has made rapid strides in the past few years. The solid biomass samples can now be pressed onto the ATR crystal at high pressures, allowing a more uniform penetration by the evanescent radiation and a higher degree of spectral reproducibility. No grinding is necessary for solid samples, facilitating ease and speed in sample preparation. More importantly, with the improved spectral data collection and reproducibility associated with the present day FTIR-ATR technique it is now possible to develop a better quality database for biomass compositional analysis. (See e.g., Tucker M P, Nguyen Q A, Eddy F P, Kadam K L, Gedvilas L M, Webb J D. 2001. Fourier Transform Infrared Quantitative Analysis of Sugars and Lignin in Pretreated Softwood Solid Residues. Appl Biochem Biotechnol 91-93:51-61.) The ATR procedure rapidly identifies the chemical changes upon pretreatment.

### Mass Balance for Washing

Approximately 6-8% and 13-15% by mass (based on dry biomass) of the untreated and AFEX treated corn stover respectively were lost in the washing step. A small fraction of the washed material was biomass fines (<0.1%) while the remainder of the extract was possibly ligno-phenolic fragments, proteins, sugars, xylooligosaccharides etc. The weight loss of the biomass samples was taken into account for the hydrolysis calculations. These results are found to be consistent with previously reported water wash data (weight loss of 12% based on dry biomass) for super/sub-critical ammonia treated birch wood. (See e.g., Chou Y C T. 1986. Supercritical ammonia pretreatment of lignocellulosic materials. Biotechnol Bioeng Symp 17:19-32; and Weimer P J, Chou Y C T, Weston W M and Chase D B. 1986. Effect of supercritical ammonia on the physical and chemical structure of ground wood. Biotechnol Bioeng Symp 17:5-18.) However, the wash extractive for ammonia treated birch wood was found to be largely acetamide (6-8% based on dry biomass) formed due to the ammonolysis of the heavily acetylated hemicellulose.

### Modified Prussian Blue (MPB) Wash Stream Characterization

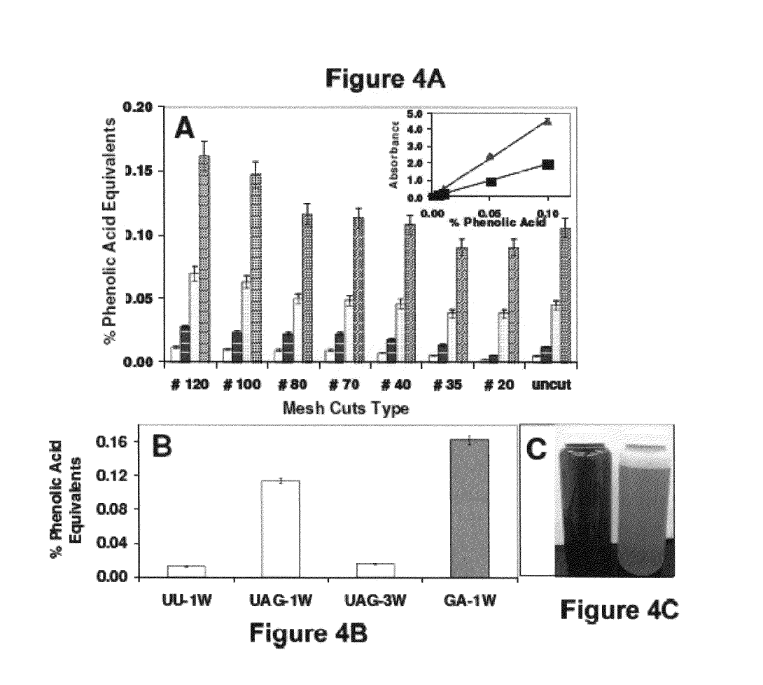

The phenolic content of the wash streams was quantified against the ferulic and gallic acid standard curves as shown with respect to FIG. 4A. The absorbance readings were highly reproducible due to better color stability compared to other colorimetric methods (See e.g., Graham H D. 1992. Stabilization of the Prussian blue color in the determination of polyphenols. J Agric Food Chem 40:801-805.)

The AFEX wash stream has nearly 6-8 fold greater phenolic content than the corresponding untreated wash stream (FIG. 4A) for the various stover fractions. The washed ligno-phenolics are likely to be from AFEX surface deposits rather than the cell wall ultra structure, given the short extraction time period (15 min) and mild extraction conditions (20° C., 1 atm). A greater proportion of phenolics are extracted from the finer fractions as opposed to the larger particle size fractions. Particle size reduction augments extraction of ligno-phenolics and other cell wall extractives during AFEX. Pre-milled corn stover was AFEX treated and milled to 0.08 mm using a centrifugal mill. The ground material was water washed to obtain the UAG-LW (Uncut PM corn stover→AFEX→Ground to 0.08 mm→Single wash) wash stream. Another sample was prepared by milling the PM corn stover to 0.08 mm, followed by AFEX. The substrate was washed to obtain the GA-1W (Ground to 0.08 mm→AFEX→Single wash) wash stream. The amount of phenolics extracted in the GA-1W wash stream was found to be greater than the UAG-1W wash stream (See FIG. 4B).

Coumaric and ferulic acid and their derivatives comprise nearly 4-6% (w/w) of the corn cell wall. (See e.g., Saulnier L, Thibault J F. 1999. Ferulic acid and diferulic acids as components of sugar-beet pectins and maize bran heteroxylans. J Sci Food Agric 79 (3):396-402.) Approximately 0.12-0.16% equivalent phenolic content was found in the AFEX wash streams; indicating roughly 10-20% of the total coumaric and ferulic acids were removed by the water washing step. Untreated stover wash stream (UU-1W: Uncut corn stover→Untreated→Single Wash) contained 10-12 fold lesser phenolic content than the AFEX treated corn stover wash streams (UAG-1W and GA-1W), as shown in FIG. 4B.

Multiple washing of AFEX treated substrates was found to remove most of the phenolic extracts from the biomass surface (FIG. 4B). The wash stream phenolic content for the triply washed substrate (UAG-3W: Uncut corn stover→AFEX→Ground to 0.08 mm→Washed three times) was 7-8 fold lesser than that of the unwashed substrate (UAG-1W and GA-1W). The glucan conversion for the once or thrice washed substrate was found to be nearly same (Data not shown). Hence, all subsequent enzymatic hydrolysis experiments were carried out for once washed substrates.

### Enzymatic Hydrolysis of Stover Fractions

### Particle Size Effect

Biomass particle size substantially affects both AFEX pretreatment and enzymatic hydrolysis. Particle size reduction increases the effective surface area to volume ratio; improving enzyme accessibility to active substrate sites. (See e.g., Mansfield S D, Mooney C and Saddler J N. 1999. Substrate and enzyme characteristics that limit cellulose hydrolysis. Biotechnol Prog 15:804-816.) Size reduction was found to have little added advantage for hot water pretreatment of corn stover (See e.g., Zeng M, Mosier N S, Goetz J, Fan P, Sherman D, Ladisch M R. 2005. Microscopic Examination of Plant Cell Structure of Enzyme-treated Corn Stover. 229th National ACS Meeting of the American Chemical Society, San Diego, Calif. Poster 335), while being deleterious for acid pretreatment due to heightened sugar degradation (See e.g., Cullis I F, Saddler J N, Mansfield S D. 2004. Effect of initial moisture content and chip size on the bioconversion efficiency of softwood lignocellulosics. Biotechnol Bioeng 85(4):413-421.) Effect of particle size on AFEX pretreatment has not been carefully investigated in the past. (See e.g., Moniruzzaman M, Dale B E, Hespell R B, Bothast R J. 1997. Enzymatic hydrolysis of high-moisture corn fiber pretreated by AFEX and recovery and recycling of the enzyme complex. Appl Biochem Biotechnol 67:113-126.) Past work on particle size reduction has largely focused on untreated or mechanically treated biomass (See e.g., Elshafei A M, Vega J L, Klasson K T, Clausen E C, Gaddy J L. 1991. The saccharification of corn stover by cellulase from Penicillium-funiculosum. Bioresour Technol 35(1):73-80), finding little added advantage to hydrolysis. A 3-6 fold size reduction (initial particle size ˜1-2 mm) of the untreated biomass is needed to see a measurable improvement in the glucan conversion. Reduction in the cellulose crystallinity upon mechanical pretreatment was reported to be a possible cause behind the improved hydrolysis. (See e.g., Chang V S, Holtzapple M T. 2000. Fundamental factors affecting biomass enzymatic reactivity. Appl Biochem Biotechnol 84-86:5-37.) However, it is more likely that milling results in enhanced conversion due to increased enzyme accessibility. (See e.g., Mansfield S D, Mooney C and Saddler J N. 1999. Substrate and enzyme characteristics that limit cellulose hydrolysis. Biotechnol Prog 15:804-816.)

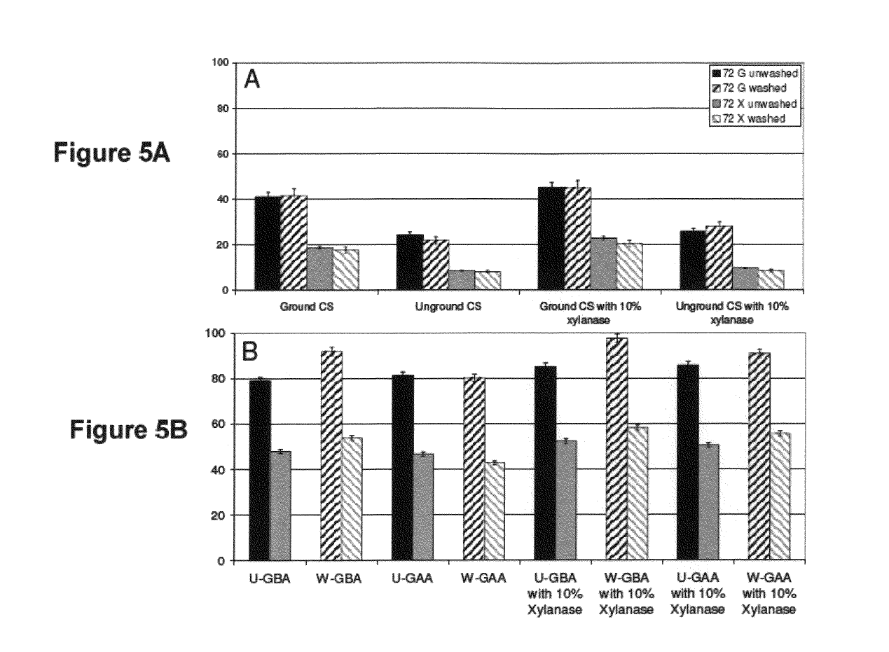

Experiments were conducted to independently elucidate the influence of particle size on enzymatic hydrolysis and AFEX pretreatment. The impact of size reduction on untreated corn stover hydrolysis was explored first. Particle size reduction (from 0.85-0.5 mm to <0.15 mm) of uncut PM corn stover enhanced glucan and xylan conversions by 15-20% as shown with respect to FIG. 5A. There was little difference in the conversions between washed and unwashed substrates. Xylanase addition was found to have a small incremental effect (<5%) on glucan-xylan conversions.

The effect of size reduction on AFEX was studied separately from its influence on enzymatic hydrolysis. PM corn stover was AFEX treated and milled to 0.08 mm using a centrifugal mill to prepare the GAA sample (GAA-Ground after AFEX). Another sample (GBA-Ground before AFEX) was prepared by milling the PM corn stover to 0.08 mm, followed by AFEX. The GM and GBA samples were water washed to remove the AFEX surface extractives. There was not much of a difference in hydrolysis yields for the unwashed GBA and GM samples as seen with respect to FIG. 5B. The unwashed GM has a slightly higher average glucan yield (1.5-2%) compared to unwashed GBA for 72-hour conversions, the difference is however more pronounced (10-12%) at 24 hours (hydrolysis data not shown). The increase was found to be statistically significant (Student's t-test with p=0.05) only for 24 hour hydrolysis data. The difference in glucan conversion is more prominent at the initial hydrolysis stage, but becomes less significant at later stages probably due to a two-pronged effect. Firstly, the amount of ligno-phenolics and other AFEX extractives have a higher surface density for the unwashed GBA compared to the unwashed GM. These AFEX extractives play an important inhibitory role in the initial stages (24 hour data) of enzymatic hydrolysis. Secondly, even though AFEX is more effective on GBA, the surface extractives inhibit the enzymes resulting in equivalent glucan conversions to unwashed GAA samples.

Washing the GBA sample (W-GBA) yielded a 10-15% increase in the glucan-xylan conversion compared to the washed GM sample (W-GAA). The enhancement in hydrolysis upon washing is possibly due to removal of enzyme inhibitory ligno-phenolic fragments. A greater amount of ligno-phenolics and other cell wall extractives to be extracted for the GBA sample as compared to the GM sample was expected, based on the modified Prussian blue results (FIG. 4B). A similar trend was seen for the xylanase supplementation experiments (FIGS. 5A and 5B). Close to 100% glucan conversion in less than 72-hours for the washed GBA samples (10% Xylanase supplemented) was observed. In general, it was shown that size reduction coupled with water washing significantly improves the hydrolysis of AFEX treated biomass.

### Compositional Effect

There was no evident trend in the hydrolysis of untreated stover fractions; with an average of <25-30% glucan (FIG. 6) and <10-15% xylan (FIG. 7) conversions. A higher conversion for the untreated #120 stover fraction was the only anomalous result. The finely ground #120 stover fraction may be comprised largely of the easily hydrolysable biomass portion (i.e. leaf, husk and sheaths), explaining the higher glucan (35-45%) and xylan (16-18%) conversions.

However, there is a clearer trend apparent for AFEX treated stover fractions. Composition of various stover fractions has an evident effect on AFEX pretreatment and subsequent hydrolysis yields. Within all AFEX treated fractions, the #20 fraction seems to be the most recalcitrant of the group probably due to higher xylan content (Table 1). The stover fractions differ appreciably in their hemicellulose content (Table 1) with relatively similar cellulose content. Fraction #20 is composed largely of corn cob granules that have a higher hemicellulose content contributing largely to its enzymatic recalcitrance compared to other fractions for the same 15 FPU cellulase loading. The commercial cellulase enzymes lack suitable hemicellulase activity resulting in lower glucan conversion for biomass rich in hemicellulose as the hemicellulosic sheath needs to be penetrated to get access to cellulosic microfibrils buried beneath. (See e.g., Fengel D, Wegener G. 1989. Wood: chemistry, ultrastructure, reactions. Walter de Gruyter: Berlin.) The glucan conversions (FIG. 6) for #20 fraction versus other fractions is 15-20% lower, on an average. However, the xylan conversion (FIG. 7) for #20 fraction is quite similar to other fractions. A possible reason being greater substrate inhibition (due to higher hemicellulose content) for #20 fraction that results in similar xylan conversions and relatively lower glucan conversions compared to other fractions. Stover fractions #35, #40 and #70 are comprised largely of plant material from the internodal region of the corn stalk, indicated by their composition and fibrous appearance under the microscope. Internodal cell walls are comprised to a large extent of highly lignified xylem vessels as compared to the non-xylem vessels. In previous work on alfalfa (See e.g., Grabber J H, Panciera M T, Hatfield R D. 2002. Chemical composition and enzymatic degradability of xylem and nonxylem walls isolated from alfalfa internodes. J Agric Food Chem 50:2595-2600), it was found that the xylem portion of the internodes was rather difficult to hydrolyze compared to the non-xylem portion. This may be due to restricted degradation of xyloglucan in the primary and secondary cell walls, due to greater interaction with lignin. Standard error bars for duplicate experiments are included in FIGS. 6A, 6B 7A and 7B. Washed biomass has larger variation in duplicates due to variability of the washing step.

### Washing Effect

Water washing the AFEX treated stover fractions gave more insight into the mechanism of AFEX, further explored using ESCA as a surface analytical tool. ESCA results show that there is a larger reduction in the oxygen to carbon (O/C) atomic ratio for finely ground (<80 microns) AFEX treated sample as compared to pre-milled (500-850 microns) samples (results not shown). This indicates that ammonia is more effective in extracting lignin and other alkaline extractives from within the biomass to its exterior surface for finely ground substrates as compared to the larger ones. These ligno-phenolic fragments and extractives deposited on the biomass surface are preferentially washed out during the wash step, enhancing the rate of enzymatic hydrolysis. The surface ligno-phenolic fragments might be responsible for enzyme inhibition hence affecting hydrolysis rate. (See e.g., Chesson A. 1981. Effect of sodium hydroxide on cereal straws in relation to enhanced degradation of structural polysaccharides by rumen microorganisms. J Sci Food Agri 32:745-758; Hartley R D. 1972. p-Coumaric and ferulic acid components of cell walls of ryegrass and their relationships with lignin and digestibility. J Sci Food Agric 23:1347-1354; and Hartley R D, Jones E C 1978. Phenolic components and degradability of the cell walls of the brown midrib mutant, bm3, of Zea mays. J Sci Food Agric 29:777-789.) However, there could be other components in the wash stream, such as organic acids (i.e. lactic acid, acetic acid) formed due to alkali degradation of holocellulose, which might equally contribute to the inhibition of enzymes. (See e.g., Sjostrom E. 1991. Carbohydrate degradation products from alkaline treatment of biomass. Biomass Bioenergy. 1 (1):61-64.) Coumaric and ferulic acids are the most potent phenolics that can inhibit carbohydrate degrading enzymes. (See e.g., Akin D E. 1982. Agron J 74:424-428; Martin S A, Akin D E. 1988. Appl Environ Microbiol 54:3019-3022; and Martin S A. 1990. Effect of phenolic components on fiber-degrading enzymes from rumen bacteria. Session 4. Proceedings of the Tri-National workshop Microbial and Plant Opportunities to Improve Lignocellulosic Utilization by Ruminants held at Athens, Ga. April 30-May 4, 1990. p-289.).

In Akin (1982), coumaric and ferulic acid retard filter paper activity (FPU) of rumen microbes at concentrations close to 0.1% was found. Martin (1990) carried out a detailed study of inhibition of ruminal and commercial enzymes by phenolic acids and found that the percentage of inhibition (5-75%) depended on the biological source of enzymes. These phenolics were also found to have potential applications as antioxidants. (See e.g., Graf E. 1992. Free radical biol med 13:435-448.) Water washing helps improve hydrolysis by removing this inhibitory effect.

Washing the AFEX treated biomass in water/weak alkali may have two important advantages to the bioconversion process; aiding enzymatic hydrolysis, and enhancing fermentability of the hydrolyzate due to removal of surface bound ligno-phenolic inhibitors. (See e.g., Jung H G, Fahey Jr G C. 1981. Effect of phenolic compound removal in vitro forage digestibility. J Agric Food Chem 29:817-820; Jung H G, Fahey Jr G C. 1983. Interactions among phenolic monomers and in vitro fermentation. J Dairy Sci 66:1255-1263; Jung H G, Fahey Jr G C, Garst J E. 1983. Simple phenolic monomers of forages and effects of in vitro fermentation on cell wall phenolics J Anim Sci 57:1294-1305; and Jung H G. 1985. Inhibition of structural carbohydrate fermentation by forage phenolics J Sci Food Agric 36:74-8.) Washing seems to consistently improve glucan conversion for AFEX treated biomass. However, washing seems to reduce xylan conversion, possibly by removing the cleaved xylooligosaccharides in the wash step along with the ligno-phenolics.

### ESCA Biomass Surface Characterization

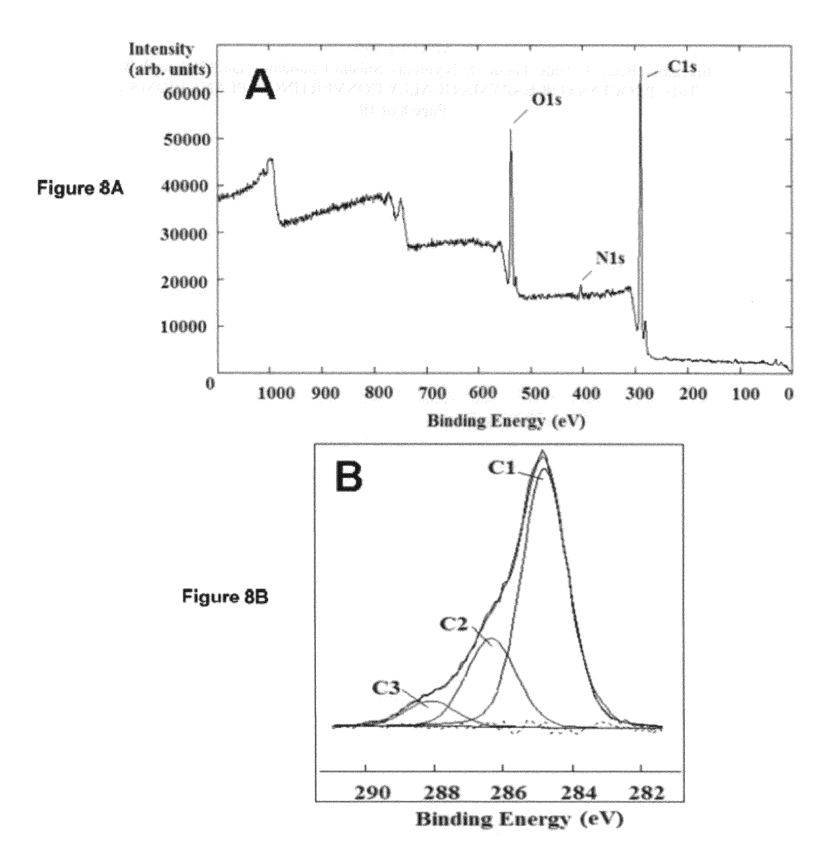

The four carbon peaks obtained upon deconvoluting the ESCA carbon spectra (FIG. 8A) belong to the following carbon bond classes; (i) C1 class of carbon that corresponds to carbon atoms bonded to carbon or hydrogen (C—C), (ii) C2 class of carbon that corresponds to carbon atoms bonded to single non-carbonyl oxygen (C—O), (iii) C3 class of carbon that corresponds to carbon atoms bonded to a carbonyl or two non-carbonyls (C═O or O—C—O), and (iv) C4 class of carbon that corresponds to carbon atoms bonded to a carbonyl and a non-carbonyl oxygen (O—C═O).

Theoretical O/C values for various cell wall constituents such as cellulose, hemicellulose, lignin and other alkali extractives can be calculated based on their empirical carbon-oxygen formula. Based on the alkyl carbon content of these components, one can place these cell wall components in a decreasing order of their O/C ratios: Cellulose>Hemicellulose>Lignin>Extractives (i.e. Fatty acids, Hydrocarbons). It becomes apparent that lignin/extractive vs. carbohydrate content on the biomass surface could be monitored by measuring the O/C ratios. Deconvoluting the carbon peak (FIG. 8B) helps to better understand the nature of the different types of carbon bonds on the surface of the biomass. In all the stover fractions only C1, C2 and C3 type carbon peaks were found. Cellulose contributes close to 85% of its signal to the C2 peak while lignin contributes 50% of its signal to C1 and the remaining signal to C2. Extractives contribute most of their signal to C1.

The surface atomic composition for various stover fractions is shown in Table 2. A drop in the C1 contribution while seeing a corresponding increase in the C2 and C3 contribution on the surface of all the fractions as compared to the PM corn stover, untreated and AFEX treated alike is seen. The AFEX process results in an increase in the carbon content on the surface of each mesh cut, while there is a corresponding decrease in the oxygen content (as depicted by drop in the O/C ratio). There is also a consistent increase in the calcium and nitrogen content on the surface of the biomass after the AFEX treatment (data not shown). It had been proposed that cell wall protein is linked to the polysaccharide through isotyrosine and diisotyrosine bridges (See e.g., Saulnier L, Thibault J F. 1999. Ferulic acid and diferulic acids as components of sugar-beet pectins and maize bran heteroxylans. J Sci Food Agric 79 (3):396-402) while calcium is thought to bind the pectin rich polysaccharides due to charged interaction with acidic side chains. These two components might be relocated to the surface of the biomass after cleavage of the lignin carbohydrate complex (LCC) during the AFEX process, hence resulting in an increase in the calcium and nitrogen signals.

TABLE 2
O/C atomic ratio and relative amounts of different carbons (C1s) of untreated (UT)
and AFEX treated (AT) corn stover and some model compounds (nd - not detected).

| | % C1 | % C2 | % C3 | % C4 | O/C | |
|---|---|---|---|---|---|---|
| Sample | UT(AT) | UT(AT) | UT(AT) | UT(AT) | UT(AT) | Reference |
| Pre-milled | 77.2 (77.2) | 17.7 (17.1) | 5.1 (5.7) | nd | 0.23 (0.20) | this study |
| # 20 (>850 μm) | 68.9 (72.2) | 24.3 (21.6) | 6.8 (6.3) | nd | 0.26 (0.22) | this study |
| # 35 (850-500 μm) | 71.5 (73.4) | 22.1 (20.6) | 6.4 (6.0) | nd | 0.25 (0.22) | this study |
| # 40 (500-425 μm) | 73.3 (75.8) | 21.0 (18.7) | 5.7 (5.5) | nd | 0.22 (0.22) | this study |
| # 70 (425-212 μm) | 73.8 (74.9) | 19.9 (19.4) | 6.3 (5.7) | nd | 0.23 (0.23) | this study |
| # 80 (212-180 μm) | 68.3 (72.1) | 24.3 (21.4) | 7.3 (6.6) | nd | 0.27 (0.23) | this study |
| # 100 (180-150 μm) | 71.3 (72.3) | 21.9 (21.0) | 6.8 (6.7) | nd | 0.25 (0.24) | this study |
| # 120 (<150 μm) | 65.4 (69.8) | 26.3 (24.0) | 8.3 (6.3) | nd | 0.28 (0.26) | this study |
| Cellulose (theoretical) | — | 83.0 | 17 | — | 0.83 | Mjoberg, 1981 |
| Bleached kraft pulp | 6 | 75 | 18 | 1 | 0.80 | Laine et al., 1994 |
| Arabinoglucuronoxylan (theoretical) | — | 78 | 19 | 3 | 0.81 | Mjoberg, 1981 |
| Xylan | 5 | 67 | 24 | 4 | 0.83 | Laine et al., 1994 |
| Lignin, Theoretical | 49 | 49 | 2 | 0 | 0.33 | Freudenberg |
| | | | | | | et al., 1968 |
| Kraft lignin | 52 | 38 | 7 | 3 | 0.32 | Laine et al., 1994 |
| Oleic acid (theoretical) | 94 | — | — | 6 | 0.11 | Dorris et al., 1978 |
| Extractives | 93 | 5 | — | 2 | 0.12 | Laine et al., 1994 |

The O/C ratio (Table 2) is a good indicator of the relative amount of oxygen rich to carbon rich species on the sample surface. Theoretical O/C ratios for some biomass components are shown in the table. A higher O/C ratio for samples rich in polysaccharides while a lower O/C ratio is seen for lignin rich samples. After the AFEX process there is a drop in the O/C ratio hence indicative of the deposition of carbon rich species on the biomass surface, i.e. lignin, proteins, extractives etc. Some experiments were conducted to determine the effect of size reduction (<80 microns) on O/C ratio as compared to PM corn stover. As expected, it was found that there is much larger drop of nearly 30% in O/C ratio for the finely ground (<80 microns) AFEX treated sample as compared to PM corn stover that sees a 10-15% drop (results not shown). These results correlate well to modified Prussian blue wash stream phenolic contents for the ground AFEX sample (GA-1W) as compared to the uncut sample (UAW-1W).

### Effect of Xylanase

As shown in Table 3, arabino-xylooligosaccharides was produced during AFEX pretreatment of corn stover and poplar extracted using an accelerated solvent extractor (ASE). These results were then quantified as acid hydrolyzed monomeric sugars (mgs per gm of original dry biomass). Some of these oligosaccharides are inhibitory for the enzymes during enzymatic hydrolysis. For hydrolyzing these oligosaccharides, sufficient activities of xylanase and xylosidase are needed in enzyme cocktail used during enzymatic hydrolysis.

TABLE 3

| | | |
|---|---|---|
| Glucose | Xylose | Arabinose |

Poplar

| | | | |
|---|---|---|---|
| AFEX treated poplar (ASE Extract) | 0.4 | 0.4 | 0.5 |
| AFEX treated poplar (ASE Extract, | 2.1 | 34.9 | 6.9 |
| acid hydrolyzed) | | | |

Corn stover

| | | | |
|---|---|---|---|
| Untreated corn stover (ASE Extract) | 24.8 | 15.8 | 1.3 |
| Untreated corn stover (ASE Extract, | 30.7 | 16.9 | 3.8 |
| acid hydrolyzed) | | | |
| AFEX treated corn stover (ASE Extract) | 12.4 | 5.3 | 1.1 |
| AFEX treated Corn (ASE Extract, acid | 27.6 | 58.3 | 20.6 |
| hydrolyzed) | | | |

With respect to Table 4 below, details related to proteomic anlysis of some particular commercial enzymes are provided. This table shows substantial activities of both endo xylanase and β-xylosidase in both Multifect™ xylanase and pectinase, which are preferred components in the enzyme cocktail in order to digest xylan component in the biomass which influences the glucan conversion.

TABLE 4

| | Glycosyl | | | | | |
|---|---|---|---|---|---|---|
| | Hydrolase | Spezyme | | Multifect | Multifect | Depol |
| | Family | CP | Novo 188 | X | P | 670L |
| Hydrolytic Proteins | | | | | | |
| Celloblohydrolase I | 7A | 334 | | | | 273 |
| Cellobiohydrolase II | 6A | 86 | | | | 138 |
| Endoglucanase I | 7B | 86 | | | | 55 |
| Endoglucanase II | 5A | 34 | 4 | | | 53 |
| Endoglucanase III | 12A | 12 | | 3 | | 9 |
| Xyloglucanase | 74 | 119 | | 132 | | 124 |
| β-glucosidase | 3 | 39 | 82 | 22 | | 18 |
| β-xylosidase | 3 | 32 | | 69 | 56 | 24* |
| Endoxylanase I | 10 | 23 | | 4 | | |
| Endoxylanase II | 11 | 11 | | 183 | | 22 |
| Endoxylanase III | 5 | 7 | | | 10 | |
| α-L-arabinofuranosidase I | 54 | 10 | | 8 | | 12 |
| α-L-arabinofuranosidase II | 62 | 9 | | 11 | 14 | 12 |
| α-glucuronidase | 67 | 9 | | 10 | 11 | |
| acetyl xylan esterase | 5 | | | 6 | | |
| Others | | | | | | |
| Swollenin | CBM I | 20 | | | | 15 |
| μ-1,4-Mannosidase | 5 | 15 | | 7 | | 12 |
| β-Galactosidase | 35 | 11 | | 15 | 23 | |
| μ-1,6-galactanase | 5 | 10 | | 14 | | |
| β-1,4-galactanase | 53 | | | | 26 | |
| Cellulose binding proteins | 1 | 43, 25 | | 24, 12 | | 29, 13 |
| Glycoside Hydrolase | 16, 30 | 5, 20 | | 19 | | |
| μ-1,3-endoglucanase | 17 | 3 | 12 | | 12 | |
| α-1,2-mannosidase | 92 | | 6 | | | |
| α-galactosidase | 31 | | 3 | | 20 | |
| Chitinases | 20 | 6 | | 6 | | |
| Endoglucanase | 61 | | | 3 | | |
| Pectate lyase | | | | | 87 | |
| Polygalacturonase | 28 | | | | 25 | 6 |
| Pectin esterase | | | | | 24 | |

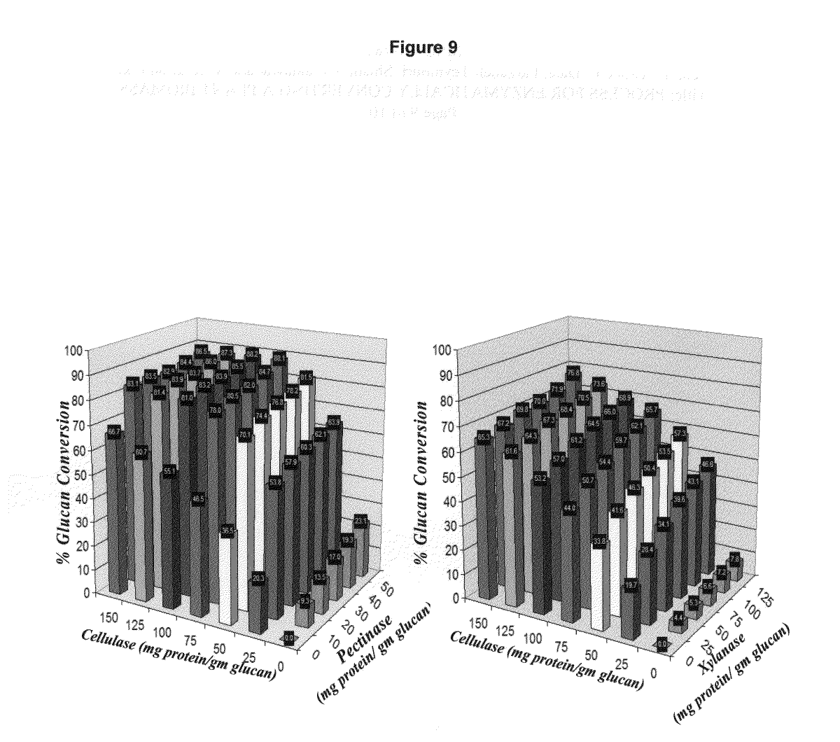

FIG. 9 illustrates, the effect of varying cellulase, xylanase (MULTIFECT X™) and pectinase (MULTIFECT P™) loading for AFEX treated corn stover [90° C., 1:1 ammonia:biomass loading, 60% moisture (dwb)]. Experiments were done using ground biomass in a micro plate with 1% glucan loading, 750 μl reaction volume, at 375 RPM. The reaction was done at 50° C. for a period of 6 hrs. As shown in FIG. 9, it is clear that both pectinase and xylanase activities along with cellulase improves glucan conversion.

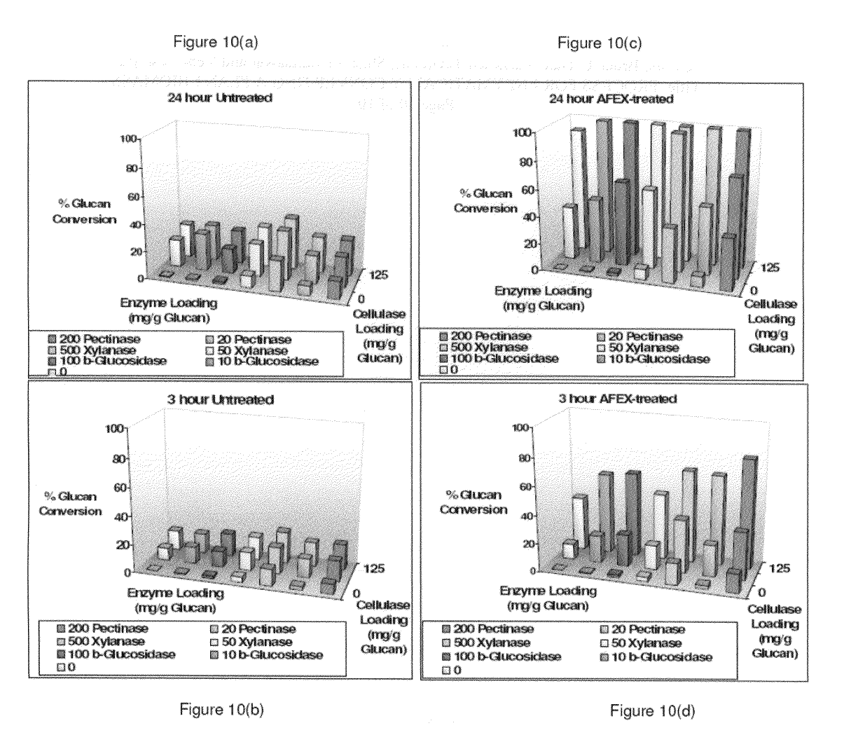

Synergistic hydrolysis of untreated and AFEX treated poplar for both 3 and 24 hrs are shown in FIG. 10. The experiments were done in a micro plate with 1% glucan loading. The enzymatic hydrolysis done at 50° C. for a period of 3 and 24 hrs respectively. It was shown that for a lower cellulase loading, both xylanase (MULTIFECT X™) or pectinase (MULTIFECT P™) improve glucan conversion.

### CONCLUSIONS

Separating the corn stover into stalk, cob and leaf rich fractions might prove fruitful in removing the more recalcitrant parts of corn stover from the less recalcitrant portions. The more recalcitrant fractions (stalk and cob rich) could be then pretreated under more severe conditions compared to the easily hydrolysable fractions (leaf-sheath rich). There is scope for optimizing AFEX conditions for each of these individual biomass fractions. This might aid in economizing the pretreatment process for corn stover, possibly reducing overall cost of bioconversion. The corn stover particle size reduction and washing were found to improve effectiveness of AFEX pretreatment and substantially improve the hydrolysis yields. The time required for complete glucan hydrolysis of milled and washed AFEX corn stover (supplemented by commercially available xylanase) was reduced by 96 hours (168 hours to 72 hours) compared to the unwashed samples.

The effect of water washing untreated/AFEX treated biomass is unclear from a fundamental (effects on cell wall ultra structure, structural elucidation of ligno-phenolics) and applied (effect of temperature/pressure, wash contact time, effect of multiple washing, enzyme inhibitory effect of extracted phenolics, fermentation inhibitors in the wash stream) perspective. Issues of recovery of xylooligosaccharides lost in the wash stream may still need to be addressed.

There is a need to evaluate the effect of water/alkali washing for AFEX treated protein-rich biomass (ex. switchgrass, distiller's grain). The effect of alkali extraction is unclear compared to simple water washing and needs to be explored further. Lignin fragments solubilized by alkali might be redeposited (See e.g., Weimer P J, Chou Y C T, Weston W M and Chase D B. 1986. Effect of supercritical ammonia on the physical and chemical structure of ground wood. Biotechnol Bioeng Symp 17:5-18) on the biomass surface, possibly reducing extraction effectiveness compared to water washing.

The ligno-phenolics formed during the AFEX process could be extracted as value-added antioxidants from the wash stream. Structural elucidation of enzyme and microbial inhibitors in the wash stream would be an important step in better understanding the pretreatment and hydrolysis system.

Sufficiently inexpensive sugars from renewable plant biomass can become the basis of a very large chemical and fuel industry, replacing or substituting for petroleum and other fossil feedstocks. Effective, economical pretreatment and hydrolysis are required to make all the potential sugars available at high yield and acceptable cost. The present invention fills this requirement.

AFEX treated corn stover is very reactive and even using only cellulase almost 95% of glucan and 75% of xylan can be converted to glucose and xylose, respectively. In this invention, cellulase and xylanase activities were traded off to achieve at least 90% total conversion of glucan and xylan to their component monomers. Process modeling by an independent research group showed that increasing the total sugar yield along with reducing the total cost of enzyme are very cost effective in the biomass conversion industry.

Potential markets that can benefit from the teachings associated with this invention include: (1) the U.S. chemical industry, which is beginning to move away from the petroleum as a source of chemical feedstocks and is interested in inexpensive sugars as platform chemicals for new sustainable processes; and (2) the fermentation industry, especially the fuel ethanol production industry which is interested in inexpensive sugars.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### NOMENCLATURE

AFEX—Ammonia Fiber Explosion
ATR—Attenuated Total Reflection
BM—Biomass
CS—Corn Stover
DRIFT—Diffuse Reflectance Infra-Red Fourier Transform
ESCA—Electron Spectroscopy for Chemical Analysis
FTIR—Fourier Transform Infra-Red
IR—Infra-Red
LCC—Lignin Carbohydrate Complex
MPB—Modified Prussian Blue
O/C—Oxygen to Carbon Ratio
PM—Pre-milled

We clam:

1. A process for converting disrupted lignocellulosic plant biomass to sugars comprising xylose and glucose, the process comprising:
(a) providing a plant biomass comprising:
(i) cell walls comprising xylans, glucans and pectins; and
(ii) glucans inside the cell walls; and
(b) adjusting a ratio of enzymatic activity of a glucanase to a hemicellulase so that at least 90% by weight of available cellulose and the xylans in the cell wall of the plant biomass are converted to the sugars.

2. The process of claim 1 wherein the plant biomass material is corn stover.

3. The process of claim 1 wherein the ratio of filter paper cellulase units (FPU) as the glucanase to units of hemicellulase is between about 10 to 1 and 2 to 1.

4. The process of claims 1 or 2 wherein the plant biomass is treated with an AFEX process step to disrupt the plant biomass.

5. The process of claims 1 and 2 wherein a reduced amount of the glucanase and the hemicellulase over an amount of each enzyme alone needed to achieve the 90% by weight conversion is used for the converting.

6. The process of any one of claims 1, 2 or 3 wherein the biomass is washed in a liquid after an AFEX process step to remove phenolics in the liquid.

7. The process of any one of claims 1, 2 or 3 with a loading of 7.5 FPU of cellulase as the glucanase and xylanase as the hemicellulase per gram of available glucans and xylans.

8. The process of any one of claims 1, 2 or 3 wherein 5 to 15 International units of xylanase as a hemicellulase and 5 to 15 filter paper units of cellulase as the glucanase are used to achieve the 90% by weight conversion.

9. The process of claim 1 wherein a weight of units of the cellulase as the glucanase with xylanase as the hemicellulase per gram of cellulose and xylan in the plant biomass is less than a weight of units of cellulase alone to achieve the 90% by weight conversion.

10. The process of claim 2 wherein the corn stover is pre-milled to reduce particle size of the biomass to be between 0.05 mm to 0.85 mm.

11. The process of claim 1, wherein step (b) further comprises adjusting a ratio of enzymatic activity by providing a pectinase in addition to the glucanase and the hemicellulase.

12. The process of claim 1 wherein the hemicellulase comprises at least one of xyloglucanase, β-xylosidase, endoxylanase, α-l-arabinofuranosidase, α-glucuronidase, and acetyl xylan esterase.

13. The process of claim 10 wherein the plant biomass is treated with an AFEX process step to expose the xylans and cellulose and then milled to a reduced particle size.

14. The process of claim 4 wherein the AFEX treatment comprises the steps of:
(a) providing the biomass with 60% moisture to a high-pressure reactor with liquid ammonia to a vessel;
(b) raising and maintaining the temperature of the vessel to 90° C. for up to five minutes; and
(c) explosively relieving the pressure to cause a pressure drop such that the ammonia vaporizes causing explosive decompression of the biomass and fiber disruption.

15. A process for converting a disrupted lignocellulosic plant biomass comprising xylan and cellulose to xylose and glucose, which comprises: adjusting a ratio of enzymatic activity of cellulase to hemicellulase so that at least 90% by weight of available cellulose and xylan in the plant biomass is converted to glucose and xylose.

16. The process of claim 15 wherein the plant biomass material is corn stover.

17. The process of claim 15 wherein the ratio of filter paper cellulase units (FPU) for cellulase to units of hemicellulase is between about 10 to 1 and 2 to 1.

18. The process of claims 15 or 16 wherein the plant biomass is treated with an AFEX process step to expose the xylan and cellulose.

19. The process of claim 15 and 16 wherein a reduced amount of cellulase with hemicellulase and an amount of cellulase alone needed to achieve the 90% by weight conversion is used for the converting.

20. The process of any one of claims 15, 16 or 17 wherein the biomass is washed after an AFEX process step to remove a liquid comprising phenolics.

21. The process of any one of claims 15, 16 or 17 with a loading of 7.5 FPU of cellulase and hemicellulase per gram of available glucan and xylan.

22. The process of any one of claims 15, 16 or 17 wherein 5 to 15 International units of hemicellulase to xylan loadings and 5 to 15 filter paper units of cellulase are used to achieve the 90% by weight conversion.

23. The process of claim 15 wherein a weight of units of the cellulase with hemicellulase per gram of glucan and xylan in the plant biomass is less than a weight of units of cellulase alone to achieve the 90% by weight conversion.

24. The process of claim 16 wherein the corn stover is pre-milled to reduce particle size of the biomass.

25. The process of claim 24 wherein the particle size is reduced to be between 0.05 mm to 0.85 mm.

26. The process of claim 25 wherein the particle size is reduced to be between 0.15 mm to 0.5 mm.

27. The process of claim 24 wherein the plant biomass is treated with an AFEX process step to expose the xylan and cellulose and then milled to a reduced particle size.

28. The process of claim 18 wherein the AFEX treatment is a pretreatment to the biomass prior to enzymatic hydrolysis comprising the steps of:
(a) providing the biomass with 60% moisture to a high-pressure reactor with liquid ammonia to a vessel;
(b) raising and maintaining the temperature of the vessel to 90° C. for up to five minutes; and
(c) explosively relieving the pressure to cause a pressure drop such that the ammonia vaporizes causing explosive decompression of the biomass and fiber disruption.

29. A process for converting disrupted lignocellulosic plant biomass to sugars comprising xylose and glucose, the process comprising:
(a) providing a plant biomass comprising:
(i) cell walls comprising xylans, glucans and pectins; and
(ii) glucans inside the cell walls; and
(b) adjusting a ratio of enzymatic activity of a glucanase to a cell-wall degrading lyase enzyme so that at least 90% by weight of available cellulose and the xylans in the cell wall of the plant biomass are converted to the sugars.

30. The process of claim 29, wherein the cell-wall degrading lyase enzyme comprises at least one of a hemicellulase and a pectinase.

31. The process of claim 30, wherein the hemicellulase comprises at least one of xyloglucanase, β-xylosidase, endoxylanase, α-l-arabinofuranosidase, α-glucuronidase, and acetyl xylan esterase.

32. The process of claim 30, wherein the pectinase comprises at least one of pectate lyase, polygalacturonase, and pectin esterase.
